(19) United States
(12) Patent Application Publication
(10) Pub. No.: US 2011/0156315 A1
(43) Pub. Date: Jun. 30, 2011

(54) System and Method for Manufacturing a Medication

(75) Inventors: Johannes Khinast, Graz (AT); Günter Brenn, Graz (AT); Andreas Zimmer, Graz (AT); Rudolf Eichinger, Graz (AT); Wolfgang Bauer, Graz (AT)

(73) Assignee: RESEARCH CENTER PHARMACEUTICAL ENGINEERING GMBH, Graz (AT)

(21) Appl. No.: 13/056,662

(22) PCT Filed: Jul. 30, 2009
(86) PCT No.: PCT/EP09/05534

§ 371 Date: Mar. 15, 2011

(30) Foreign Application Priority Data
Jul. 30, 2008 (EP) 08013696.3

(51) Int. Cl. B29C 39/36; B05C 5/00; B05C 11/00; A61K 9/28; B29C 53/00

(52) U.S. Cl. 264/334; 118/300; 118/696; 118/713; 427/2.14; 264/239

(57) ABSTRACT

An apparatus for manufacturing a medication comprising an ejector unit adapted for ejecting a predefined amount of a drug having a liquid component to a solid carrier substrate. The ejector unit comprises a capillary and a tubular piezoelectric actuator surrounding at least a part of the capillary. The apparatus further comprises a control unit adapted for applying an electric signal to the piezoelectric actuator which, in response to the electric signal, is adapted to generate a compressional wave in the capillary for ejecting the predefined amount of the drug via an orifice of the capillary. Moreover, a method of manufacturing a medication is provided, the method comprising ejecting a predefined amount of a drug having a liquid component to a solid carrier substrate. Furthermore, a medication is provided comprising a solid carrier substrate, and a predefined amount of a drug ejected with a liquid component to the solid carrier substrate by an ejector unit.

### CROSS REFERENCE TO RELATED APPLICATIONS

This application claims the benefit of the filing date of the European Patent Application No. 08013696.3 filed Jul. 30, 2008, the disclosure of which is hereby incorporated herein by reference.

### TECHNICAL FIELD

The invention relates to an apparatus for manufacturing a medication.

Moreover, the invention relates to a method of manufacturing a medication.

Beyond this, the invention relates to a medication.

### BACKGROUND

Medication, also referred to as medicine, is usually a drug or any other substance used to prevent or cure disease or to relieve pain or any form of perceived discomfort. Conventionally, medication is administered to a patient for instance in the form of pills, tablets, suppository or as a liquid.

EP 1,306,071 A2 discloses a method of manufacturing a bioactive fluid dose on an ingestible sheet, comprising the steps of advancing the ingestible sheet to a dispense position, and activating a fluid ejector to dispense essentially a drop of a bioactive fluid onto the ingestible sheet.

US 2005/233000 A1 uses a jettable pharmaceutical solution. US 2005/233000 A1 discloses a method for producing an oral medication which includes dispensing a structural material, the structural material including one of a polymer or a gelatin, curing the structural material, and dispensing the jettable pharmaceutical solution onto the cured structural material.

WO 2003/005950 A2 discloses an apparatus and a method for manufacturing a pharmaceutical dose which dispenses a variable selectable quantity of at least one pharmaceutical onto a pharmaceutical receiving medium. The quantity of the dispensed pharmaceutical(s) are controllably dispensed to customize each pharmaceutical dose to suit the needs of a particular user. The apparatus is coupled by an external telecommunication network to a remote signal source for receiving pharmaceutical quantity and type data for custom manufacturing a pharmaceutical dose. In one aspect, a replaceable cartridge contains a reservoir carrying at least one pharmaceutical component and a fluid drop generator which is mountable in the fluid dispenser. The reservoir may contain a number of separate compartments, each carrying a different pharmaceutical component.

WO 2005/009738 A2 discloses a method of controlling a dissolution rate of a bioactive agent which includes selecting a desired dot topography corresponding to a target dissolution rate and applying a bioactive agent to a delivery substrate to form dots having the desired dot topography on the delivery substrate.

DE 199 20 241 A1 discloses a method for machining platelike workpieces in a striated manner, whereby, in work steps, the material plate is machined by a controlled linear unit in a striated manner and always in the same X-axis direction.

WO 2004/009445 A2 discloses an oral dosage delivery vehicle comprising an edible film including a uniformly distributed active ingredient, wherein said film comprises dosage units releasably joined by one or more weakened sections, which permit said dosage units to be detached from said film.

GB 464,884 discloses a method of medicating sheets or webs of paper, fabric or like materials, or sheet-like articles made of such materials, which consists in applying to the sheets, webs or articles a liquid medicament capable of visibly marking them by means of a rotary cylinder having an embossed surface to which the medicament is supplied and which is adapted to mark the whole or substantially the whole of the surface of the sheets, webs or articles with a close pattern.

However, conventional systems may lack sufficient performance.

### SUMMARY

It is an object of the invention to provide an efficient way of manufacturing a drug to be administered to a patient.

In order to achieve the object defined above, an apparatus for manufacturing a medication, a method of manufacturing a medication, and a medication according to the independent claims are provided.

According to an exemplary embodiment of the invention, an apparatus for manufacturing a medication is provided comprising an ejector unit adapted for ejecting a predefined amount of a drug having a liquid component to a solid carrier substrate.

Optionally, the ejector unit comprises a capillary and a tubular piezoelectric actuator surrounding at least a part of the capillary. Optionally, the apparatus further comprises a control unit adapted for applying an electric signal to the piezoelectric actuator which, in response to the electric signal, is adapted to generate a compressional wave in the capillary for ejecting the predefined amount of the drug via an orifice of the capillary. However, alternative ejection, particularly printing and/or metering, technologies may be applied as well according to other exemplary embodiments of the invention. Thus, piezoelectricity is only one preferred embodiment, but not the only possible one. Particularly, all embodiments described herein and not being unambiguously limited to piezotechnology may also be applied to an apparatus (and to a method and to a medication) implementing or using an ejector mechanism which does not use piezotechnology.

According to another exemplary embodiment of the invention, a method of manufacturing a medication is provided, the method comprising ejecting a predefined amount of a drug having a liquid component to a solid carrier substrate.

Optionally, a piezoelectric actuator may be provided surrounding at least a part of a capillary. Further optionally, a predefined amount of a drug having a liquid component may be ejected to a solid carrier substrate by applying an electric signal to the piezoelectric actuator so that the piezoelectric actuator, in response to the electric signal, generates a compressional wave in the capillary to thereby eject the predefined amount of the drug via an orifice of the capillary.

According to still another exemplary embodiment of the invention, a medication is provided comprising a solid carrier substrate and a predefined amount of a drug ejected with a liquid component to the solid carrier substrate by an ejector unit.

Optionally, such a medication may be manufactured by an apparatus having the above mentioned features or by a method having the above mentioned features.

The term “physiological object”, to which a medication may be administered, may particularly denote any human being, any animal, and any plant (any organism).

The term “physiologically active substance”, which may be part of a medication to be administered, may particularly denote any substance which may have an effect on the physiology of the living organism. In contrast to this, the term “physiologically inert substance” may particularly denote any substance which may be free of causing any effect on the physiology of a living organism.

The term “biocompatible” may particularly denote a material property of a substance, namely that the substance, when inserted in living tissue, does not harm or negatively influence the physiological conditions at such a location in a body.

The term “dispenser device” may particularly denote any device for emitting or applying any substance to a specific region in space, particularly onto a defined surface portion of a substrate.

The term “medication” may be denoted as a substance to cure or reduce symptoms of an illness or a medical condition or to relieve pain or any form of perceived discomfort. A medication or medicine may be something that treats or prevents or alleviates symptoms of a disease.

The term “drug” may be denoted as any physiologically active substance. It may denote any substance or pharmaceutical product for human or veterinary use that is intended to modify physiological systems or pathological states for the benefit of the recipient. In the present application, the term drug may particularly denote the active pharmaceutical ingredient which is intended to be administered to the physiological subject for the medical purposes.

The term “ejector unit” may particularly denote an entity which is capable of accommodating a drug substance and for ejecting, spraying, dispensing, etc. a predetermined amount of a drug onto a predetermined surface portion of a solid carrier substrate. Such an ejector unit may maintain a distance between an ejection opening releasing the drug on the one hand and a surface of the solid carrier substrate on the other hand.

The term “solid carrier substrate” may particularly denote an underlie or a carrier for receiving the drug. The solid carrier substrate itself may be physiologically inert, i.e. may not contribute as an active pharmaceutical ingredient when the manufactured medication is administered to a physiological subject. For example, the solid carrier substrate may be made of a biocompatible material, particularly of a material which does not have any negative physiological impact on a physiological subject when the latter incorporates the solid carrier substrate. An example for an edible solid carrier substrate is a wafer as may be used as a consecrated wafer for religious procedures. Particularly, the solid carrier substrate may be formed on the basis of cellulose. Cellulose may be a polysaccharide of beta glucose which may form the primary structural component of green plants. Other complex carbohydrates or polysaccharides, etc. can be used as well as a solid carrier substrate. It is possible that the solid carrier substrate is made of a substance or comprises a substance which a human body can metabolize or digest.

The term “piezoelectric actuator” may particularly denote a member showing the piezoelectric effect which describes the relation between a mechanical stress and an electrical voltage (or an electrical current) in solids. In piezoelectric actuators, an applied voltage will change the shape of the solid by a small amount. This effect can be used by exemplary embodiments for generating a pressure within a capillary to enable accurate ejection of a droplet of a drug containing liquid.

According to an exemplary embodiment of the invention, a medication manufacturing system as well as a correspondingly manufactured medication are provided, wherein an ejection unit ejects predetermined amounts of drugs having a liquid component (such as water or any other solvent) onto a specific portion of a solid carrier substrate. By taking this measure, it is possible to accurately manufacture, on an industrial scale, individual medication doses with individually definable properties. Thus, each piece of manufactured medication may be specifically configured to the needs of an individual patient regarding parameters such as dose, kind of medication, mix of different physiologically active substances, etc. Different portions of the solid carrier substrate—each of which may be administered to a physiological subject—may be provided simultaneously or sequentially with specific amounts of physiologically active substances. The accuracy of the definition of the drug applied to each of the segments of the solid carrier substrate is very high due to the usage of an ejection procedure. Embodiments of the invention allow to provide an individual therapy in a patient-specific manner.

According to an exemplary embodiment of the invention, the ejection may be realized by a concentric arrangement of a tubular capillary and a tubular piezoelectric actuator. Thus, it is possible to apply a tubular compression (or expansion) force onto the capillary portion covered by the piezoelectric hollow cylindrical structure, thereby allowing for a very precise control of the fluidic properties within the piezoelectric tube. For instance, a tubular outer surface of the capillary may be constricted by a contracting impact of the activated piezoelectric actuator. When a corresponding electric signal is applied (for instance via appropriately located electrodes) to the piezoelectric actuator, a contracting force may be impacted via the actuator onto the tubular capillary section, thereby generating a pressure pulse acting on a spatially extended portion of the capillary. Thus, particularly in the field of pharmacy where a very precise control of an amount of drug to be ejected has to be ensured, such an architecture is highly advantageous.

Next, further exemplary embodiments of the apparatus will be explained. However, these embodiments also apply to the method and to the medication.

Such a piezo-based ejection mechanism may be performed under consideration of the boundary condition in pharmatechnology that such an apparatus and corresponding operation method should be adapted to operate in accordance with Good Manufacturing Practice (GMP). It should be configured for use in a sterile environment. Hence, it may be advantageous that exclusively inert materials are used for all components of the apparatus coming into contact with pharmaceutical agents in order to avoid any kind of contamination, impurity or other negative impact. Solvents which may be used for metering pharmaceutical agents may have specific properties regarding surface tension, viscosity and/or polarity which should be considered when selecting materials of the apparatus. Not only the use of specifically selected materials, but also a geometric configuration of the ejection nozzle should be adapted to promote droplet formation of the described materials at an outlet of the nozzle. Furthermore, drive voltages, pulse lengths and other parameters related to a piezoelectric ejection mechanism may be adjusted to meet the above boundary conditions. Particularly to be in accordance with GMP and/or Food and Drug Administration (FDA) boundary conditions, the apparatus may be provided with a documentation unit (which may include one or more of an RFID system, a camera, an optical sensor for counting number and/or volume of droplets, measurement of a number of pulses, etc.). For specific pharmaceuticals, the use of coolable and/or light-protected or light shielded containers may be advantageous.

The capillary may be circumferentially surrounded, along a jacket at the cylindrical outer perimeter of the capillary, by the tubular piezoelectric member.

The capillary may have a tubular section at least partially covered with the piezoelectric actuator and may have a tapering end section forming the orifice and being free of the piezoelectric actuator. In an embodiment, the capillary may be shaped in a tubular way and may have a tapering end section forming the orifice. Therefore, a tube which may form a main part of the longitudinal extension of the capillary can be simply reduced in diameter in an end section thereof, thereby forming a nozzle which allows to properly define a destination of an ejected droplet and which may, in an operation state in which no pressure is applied to the capillary, may also generate sufficiently high capillary forces to prevent a droplet from being ejected from the nozzle.

The apparatus may comprise a first electrode and a second electrode, wherein the piezoelectric actuator is arranged between the first electrode and the second electrode, and wherein the control unit is adapted for applying the electric signal between the first electrode and the second electrode. In an embodiment, the apparatus may comprise a first electrode and a second electrode electrically coupled to the piezoelectric actuator. The piezoelectric actuator may be sandwiched, i.e. arranged, between the first electrode and the second electrode. The control unit may be adapted for applying the electric signal between the first electrode and the second electrode, thereby impacting the piezoelectric actuator. Hence, two tubular electrodes which may be realized as metallization layers may be formed to cover opposing cylindrical surface portions of the tubular piezoelectric actuator. Such an arrangement may be manufactured in a simple way and may allow to properly control a compression/an expansion along the longitudinal extension of the piezoelectric hollow cylindrical structure and, in turn, of the hollow cylindrical capillary. Therefore, such a surrounding geometry may allow to precisely define the drug ejection properties of the system.

Each of the first electrode and the second electrode may be formed by a tubular metallization, wherein the tubular metallizations are applied onto opposing surfaces of the piezoelectric actuator. According to an exemplary embodiment, each of the first electrode and the second electrode may be formed by a tubular metallization layer, wherein the tubular metallization layer may be applied or deposited onto opposing surfaces of the piezoelectric actuator. In such an embodiment, electric connections to the control unit may be provided capable of applying an electric trigger signal, for instance a high voltage pulse, to the piezoelectric actuator for precisely defining the drug amount ejected as a droplet via the orifice.

Furthermore, the apparatus may comprise a temperature adjustment unit adapted to control a temperature of the liquid comprising drug before ejection of the liquid comprising drug through the orifice. Such a temperature adjustment unit may particularly be a heat application unit adapted to apply heat to the liquid comprising drug before leaving the capillary. Therefore, it is possible to precisely control the viscosity of the fluid before injection, thereby also allowing to properly adjust the fluid flow properties before (and after) ejection. In an embodiment, it may also be possible that also cooling of the liquid is enabled, for instance in a scenario in which the temperature of the fluid is too high for a proper ejection. In this scenario, a cooling unit such as a Peltier element can be used as the tempering unit. Such a Peltier element may also be used for a heating. In a scenario, in which only heating is required, an ohmic heating element may be provided such as a coil spirally wound around the capillary and/or around an optional fluidic conduit between the capillary and a fluid container. Additionally or alternatively, an ohmic heating element may be provided for heating fluid within a fluid container (such as a coil spirally wound around a fluid container). Such a temperature adjustment unit, particularly heating element, may also be arranged within the capillary and/or the conduit.

In an embodiment, the temperature adjustment unit, particularly the heat application unit, may be adapted to apply heat to the liquid comprising drug before leaving the capillary. Therefore, already in a state in which the fluid is properly controllable, i.e. within the capillary, the temperature adjustment may be performed. Then, also the viscosity properties which are of relevance with regard to the fluid properties when leaving the capillary can be properly controlled. By pre-heating the solution (a solution may be denoted as a homogeneous mixture of two or more substances, frequently (but not necessarily) a liquid solution, wherein in a solution, one or more solutes may be dissolved in another substance, which may be denoted as a solvent) or suspension (a suspension may be denoted as a liquid with particles (particularly solid particles) suspended in it), the viscosity may be reduced and the surface tension may be reduced to achieve a faster drying of the drug containing liquid. This may increase the speed of manufacturing the medications, and may also be of relevance for some drugs which have to be dried quickly in order to maintain their physiological function.

According to an embodiment, the control unit may be adapted for applying a high voltage pulse as the electric signal to the piezoelectric actuator. Such a short and very intense pulse may allow to abruptly compress the capillary in a section surrounded by the piezoelectric tube which allows for a precise and strong pressure pulse. Such a pulse may have a duration between 1 μs and 500 μs, particularly between 5 μs and 160 μs. Other durations of pulses are possible as well, so that the scope should not be limited by the given examples.

The capillary may comprise at least one material of the group consisting of a metal, a ceramic, plastic, and glass. The capillary may be made of various materials such as a dielectric material, a ceramic material, glass, or a plastic material. Particularly suitable is a material (such as glass) capable to withstand the high mechanical impacts acting or being exerted on such components when the piezoelectric pulse is applied.

According to an exemplary embodiment, the capillary may have a tubular shape and may be in fluid communication with one or more drug containers via one or more continuous or bifurcated fluidic conduit(s). In such a configuration, one or several drug containers may be brought in fluid communication with the capillary so that fluid can be sucked from one or several of these drug containers simultaneously and can be supplied via the fluidic conduit to the capillary. In an embodiment, the pressure pulse generated by the piezoelectric actuator alone may be sufficient to pump the drug containing liquid from the fluid container via the fluidic conduit into the capillary. This may allow to render a separate pump dispensable which allows for a small and efficient configuration. However, in another embodiment, such a pump may be provided in the fluidic path between container(s) and capillary.

According to an exemplary embodiment, the apparatus may be configured to be in accordance with Good Manufacturing Practice (GMP), particularly in accordance with current Good Manufacturing Practice (cGMP). Particularly, the apparatus may be in accordance with or may be certified in accordance with EC Directive 2003/94/EC. It may also be possible that the apparatus is in accordance with US requirements regarding GMP, particularly fulfils the requirements defined by the Food and Drug Administration (FDA). Particularly, the GMP regarding the manufacture of sterile medications should be fulfilled by the apparatus. GMP requires in a holistic approach that manufacturing and laboratory testing environment has to be regulated to meet specific quality requirements. A part of GMP may require documentation of every aspect of the process, activities and operations involved with drug and medical device manufacture. Such a documentation may be achieved, for instance, by using barcode technology or RFID technology. Thus, the apparatus may be capable to provide for a documentation showing how a drug was made and tested (which enables traceability and, in the event of future problems, recall from the market). In an embodiment, the requirement of GMP accordance may include that all manufacturing and testing equipment has been qualified as suitable for use and that all operational methodologies and procedures (such as manufacturing, cleaning and analytical testing) utilized in the drug manufacturing process have been validated (according to predetermined specifications) to demonstrate that they can perform their purposed functions.

Particularly, washing and cleaning equipment should be chosen and used in order not to be a source of contamination. Parts of the production equipment coming into contact with the product should not be reactive, additive or absorptive to such an extent that it will affect the quality of the product and thus present any hazard. Measuring, weighting, recording and controlling equipment should be calibrated and checked at defined intervals by appropriate methods. Adequate records of such tests should be maintained, for instance stored, by the apparatus.

Particularly, the apparatus should be configured for operation in a sterile environment. All components of the apparatus coming into contact with at least one of the drug and the solid carrier substrate should be made of an inert material. “Inert” may particularly denote that any undesired chemical reaction or physical influence of solid carrier material and drug material should be prevented.

According to an exemplary embodiment, the apparatus may further comprise a compression and encapsulation unit adapted for compressing a size of the solid carrier substrate after ejection of the predefined amount of the drug thereon and adapted for encapsulating the compressed solid carrier substrate within a capsule. Such a capsule may for instance be a gelatine capsule (or any other physiologically inert and digestible inert shell). Such a capsule may comprise a first and a second part both defining an accommodation volume and being capable of being connected to one another so as to sealingly enclose a medication such as a part of the solid carrier substrate being covered with the drug. In this context, the term “compressing” may particularly denote a reduction of the size of the solid carrier substrate carrying the medication. For example, such a size reduction may be achieved by folding the solid carrier substrate, i.e. by defining one or more folding lines according to which the solid carrier substrate can be folded so that various layers are provided one above the other. In an alternative embodiment, the compression may also include rolling of the planar carrier substrate so as to define some kind of roll which can then be accommodated within a medication capsule. Taking this measure may allow to provide the medication in an administrable form or formulation so that the capsule may be simply administered to a patient. By inserting a rolled paper strip in a gelatine capsule, it is possible to manufacture the medication in a simple manner, since this does not require any special machine or the use of powder (as in case of a tablet).

In an embodiment, a perforation of the solid carrier substrate, for instance edible paper, may be flexibly defined during the manufacturing process by a separation unit (such as a cutting unit) at the apparatus. Therefore, the dimensions of the solid carrier substrate to be separated from the remainder thereof can be defined in accordance with user or application-specific requirements. For instance, in a hospital, some patients may require only a small number, for instance only a single, drug. Other patients may require a mixture of several drugs, for instance a mixture of five or ten drugs. In accordance with the number of drugs or the amount of drugs required for a specific patient, the required dimension of a portion of the solid carrier substrate to be separated may be selected. This allows for a flexible use of the apparatus.

The ejector unit may comprise at least one of the group consisting of a nozzle, a pipette, and a needle. The ejector unit may comprise a nozzle. Via such a nozzle, one or more droplets of the drug may be ejected onto a specific portion of the substrate. Alternatively, a pipette may be used in which a specific amount of drug is injected, and, operated manually or automatically, a precisely definable volume of the drug may be spotted onto a specific portion of the substrate. It is also possible to use a sampling needle having a lumen through which the drug is injected onto the surface of the substrate. Also a capillary may be used which can be activated by pressure pulses so as to eject a number of droplets having a defined volume each onto the surface of the substrate.

In an embodiment, it is possible to use a long stripe of solid carrier substrate which is moved continuously along a transport direction (for instance using a conveyer belt or the like) so that sections of the solid carrier substrate subsequently pass the ejector unit for drug deposition. The ejector may then, for instance, move in one or two directions perpendicular to a moving direction of the solid carrier substrate. Such a motion may be vertical or horizontal. In another embodiment, the solid carrier substrate may be maintained fixed in space, and the ejector unit may move in two or three dimensions so as to dispense the drug onto a desired surface portion of the solid carrier substrate.

In an embodiment, the apparatus comprises a drug container adapted for accommodating the drug. A conduit may be provided and adapted for providing a fluid communication between the drug container and the ejector unit for conducting the predefined amount of the drug from the container through the conduit towards the ejector unit. Such a drug container may comprise drug material for a large number of medications to be manufactured. During the manufacturing procedure, the drug material may be conducted towards the ejector unit via a conduit such as a tube. Such a configuration may allow to keep the number of moving components small.

In another embodiment, the ejector unit may load the drug to be subsequently applied to the solid carrier substrate from a drug container by driving towards the drug container, immersing into the container, sucking drug material into the ejection unit, driving the ejection unit towards the solid carrier material and subsequently ejecting the drug to the solid carrier material. In such a movable configuration, the movability of an ejection unit relative to the solid carrier substrate can be used to flexibly eject the drug onto each desired surface portion of the solid carrier material.

The ejector unit may be adapted for simultaneously ejecting a predefined amount of a drug to different portions of the solid carrier substrate. Thus, in accordance with a specific sequence, the ejector unit moves relative to the solid carrier substrate and ejects constant or varying amounts of the drug to the different portions of the solid carrier substrate.

A volume of a drug to be applied to the solid carrier substrate may, in an embodiment, range from 1 pl to 10 ml (for instance may be tens of picolitres), particularly may range from 1 nl to 1 ml. Other volumes are possible.

Additionally or alternatively, the ejector unit may be adapted for ejecting predefined amounts of a plurality of different drugs to different or identical portions of the solid carrier substrate. Thus, a multi-drug comprising medication may be formed which has a plurality of different physiologically active substances. These may be applied one after the other or simultaneously onto different portions of the solid carrier substrate, or on the same portion. This may allow to design even complex pharmaceutical medications having multiple pharmaceutically active components for individual therapy.

The ejector unit may be adapted for ejecting a predefined amount of a drug having a liquid component into and/or onto a solid carrier substrate. The ejector unit may be adapted for ejecting a predefined amount of a drug having a liquid component into the solid carrier substrate. Thus, the drug may be injected into an interior of the solid carrier substrate, for instance to adjust a delayed release of a specific pharmaceutically active agent after administration to a patient. In such an embodiment, the use of an absorbent or an absorptive solid carrier substrate may be advantageous.

Additionally or alternatively, the ejector unit may eject the predefined amount of the drug onto a solid carrier substrate. In such an embodiment, the solid carrier substrate may be impermeable for the drug so that, by stacking multiple layers of the solid carrier substrate above one another, individually designed medications with different pharmaceutically active components may be formed, wherein a sequence of releasing the various drugs to a physiological object incorporating the medication may be adjusted by a position of a specific drug within a layer stack. For example, when a multi-layer medication is administered to a patient, and the patient starts digesting the exposed layers, one layer after the other will be decomposed by digestion. In accordance with this, a precisely determinable delayed release of the individual drugs may be adjusted. Thus, a spatial distribution of individual drugs may result in a corresponding temporal sequence of releasing the respective drug components.

The ejector unit may be adapted for applying the drug to the solid carrier substrate by printing. Such a printing technology may be performed using a print head which ejects a specific portion of the drug in a similar manner as in ink-jet technology. Thus, droplets of the drug may be ejected via a nozzle of such a printing head, allowing for a precise (regarding volume and position) adjustment of the medication to be administered.

The apparatus may comprise a control unit adapted for controlling the ejector unit in accordance with a predefined or user-defined protocol. For example, a fixed protocol may be adjusted for manufacturing a standard medication. Alternatively, individual prescriptions from a doctor or physician may be programmed into the control unit allowing to manufacture user-defined drugs with a printing protocol which is brought in accordance with the user's needs. A possible field of application of such an embodiment is a hospital or a pharmacy. This may allow to precisely adjust a specific amount of a drug in accordance with a body weight of a person, a specific mix of different medications to a specific disease or to a desired therapy, etc. It is also possible to manufacture a medication for an individual case in accordance with a user-defined protocol which may be defined by a physician. For example, embodiments of the invention may be implemented in a large medication production plant manufacturing medications for standard use. Alternatively, the system may also be implemented in a hospital or in a doctor's practice or in a pharmacy to manufacture medications specifically matching with user requirements.

The control unit may be adapted for controlling one or more of the parameters of a kind of a drug, an amount of a drug, a position to which the drug is to be ejected to the solid carrier substrate, and/or a geometrical shape of the applied drug. These and other parameters may be used as design parameters for achieving accordance between a user's need and the manufactured drug.

The ejector unit may be adapted for ejecting the drug to a selectable position of the solid carrier substrate.

The apparatus may comprise a monitoring unit for monitoring the predefined amount of the drug ejected onto the solid carrier substrate. Additionally or alternatively, the monitoring unit may monitor other parameters than the amount of ejected drug, for instance a proper spatial position of the drug ejection to the solid carrier substrate, etc. For example, such a monitoring unit may be an optical monitoring unit such as a camera (which may comprise a CCD, a CMOS array, etc.). Such a monitoring unit may monitor a spot of a drug administered by the apparatus and may determine whether the administered drug meets predefined requirements within predefined tolerances. If this is the case, the corresponding medication is approved to be subsequently administered to the patient. If this is not the case, the corresponding medication item may be classified as rejection/waste. Such defective goods may be excluded from being subsequently administered to a patient. Alternatively, for instance when the monitoring unit determines that the amount of ejected drug is too small, it is possible that a printing head of the ejector unit is guided again towards the drug deposition position to provide the lacking amount of drug. An output signal of the monitoring unit may be analyzed using automatic image processing routines in order to take a decision whether a manufactured medication is appropriate or not. As an alternative to an optical monitoring, it is also possible to introduce a UV-based monitoring procedure or an infrared-based monitoring procedure.

The printing procedure can be adapted in such a manner that, by the continuous analysis of the drug amount, after manufacture of a single dose the application of the drug may be interrupted, and subsequently the production parameters which may include information regarding the drug content and the manufacture may be printed on the paper. Subsequently, the application of the next single dose may be performed.

The apparatus may comprise a singularization unit adapted for a singularization of the solid carrier substrate into separate sections each comprising a drug. Such a singularization unit may divide or separate the solid carrier substrate into different medication units. Such a separation may be performed in a manner that the individual medication units are completely separated from one another, i.e. have no physical (more precisely: no mechanical) connection any more.

Alternatively, it is possible that the singularization is only partial, for instance perforations are formed between adjacent medication items. By providing perforations, it is possible to manufacture a medication which can be rolled up so that a physician can selectively detach or separate one or more pieces or units of the medication from the roll (for instance in a similar manner as with a kitchen roll). This may allow a physician, with a single hand movement, to provide and define an appropriate dose for a patient.

The apparatus may comprise an identifier forming unit adapted for forming (particularly printing) an identifier on the solid carrier substrate indicative of the ejected drug or characterizing the medication and/or the manufacture procedure. Such an identifier may be printed onto the solid carrier substrate and may include information regarding the drug, the manufacturing procedure, a date of manufacture, an expiry date of the medication, an identification of a patient (for instance name) to whom the medication is to be administered, etc. Such a procedure may later allow to accurately reproduce or retrace the history of the medication manufacture. In an embodiment, it is possible that the identifier is formed by the drug itself, i.e. the drug is administered in accordance with a specific pattern which does not only serve as a physiologically active substance to be administered to a patient, but also includes an inscription, a bar code, an alphanumerical code, etc. allowing to derive the manufacturing information from the identification. It is also possible that the identification is printed with ink or the like onto the solid carrier substrate. It is also possible that the identification is formed by correspondingly perforating the solid carrier substrate.

The apparatus may comprise a drying unit adapted for drying the drug after provision to the solid carrier substrate. The drug may be applied at least partially in a liquid form onto the solid carrier substrate. The drug may consist of liquid material or may comprise, in addition to a liquid phase, further components in other phases (solid, gases, etc.). In an embodiment, a solvent (such as water or an alcohol) may be added to the drug before ejection to the solid carrier substrate. A drying unit may, after deposition of the drug to the solid carrier substrate, dry the drug by removing at least a part of the liquid component. For example, such a drying unit may comprise a ventilator drying the drug spot by a stream of a gas such as air. Additionally or alternatively, heat may be applied to the liquid comprising drug to promote evaporation of the liquid component or a part thereof, as a drying procedure. After the drying, the drug is properly fixed at a specific location of the solid carrier substrate.

The drying unit may be configured for drying the drug after provision to the solid carrier substrate by irradiation of the drug by electromagnetic radiation, for instance using infrared radiation or radiation of any other suitable wavelength.

In addition to the drug, one or more auxiliary substances may be added to the drug by an auxiliary substance provision unit. Such an auxiliary substance provision unit may include one or more further print heads for providing additional substances such as an agent for modifying a release of an active component of the drug, an agent for modifying a degradation of the solid carrier substrate (for instance, a substance by which a degradation characteristic—for instance a digestion time—of the substrate within a body of a human being may be adjusted—for instance delayed), an agent for increasing a stability of an active component of the drug, etc. During the production procedure, printing of one or more auxiliary substances is possible. Possible applications are the modified release of an active component, a modified degradation, an increase of the stability of the medication by adding an antioxidant, reflectors for the prevention of influences from irradiated light, etc.

Next, further exemplary embodiments of the method will be explained. However, these embodiments also apply to the apparatus and to the medication.

The method may comprise providing the drug as one of the group consisting of a solution, a suspension, an emulsion, a molecular dispersive substance, a colloid dispersive substance, and a coarse dispersive substance.

The method may comprise providing the drug as a solution. A solution may be denoted as a homogeneous mixture of two or more substances, frequently (but not necessarily) a liquid solution. In a solution, one or more solutes may be dissolved in another substance, which may be denoted as a solvent.

It is also possible that the drug is provided as a suspension. A suspension may be denoted as a liquid with particles (particularly solid particles) suspended in it. In other words, a solid may be suspended in a liquid bath to form a suspension.

It is also possible that the drug is provided as an emulsion. An emulsion may be denoted as a suspension of particles of one liquid in another liquid which do not dissolve in each other. In other words, an emulsion may be a mixture of two or more liquids that are not soluble in one another.

The drug may be ejected to an endless (or quasi-endless) solid carrier substrate. For example, a roll of a long solid carrier substrate may be manufactured continuously or may be wound up from a source roll. It may then be guided along the ejector unit for provision of the drug, and afterwards may be wound or coiled onto a destination roll. Thus, a quasi continuous and therefore highly efficient manufacturing procedure may be used.

In an embodiment, it is possible that a shape of the medication is deformed after ejecting the drug to the solid carrier substrate. After having supplied a mechanically flexible or bendable substrate with a desired amount of drug, it is possible to fold or roll up such a flexible material in order to change the shape thereof. For example, some kind of pill or tablet may be formed by changing the shape of the essentially two-dimensional planar carrier substrate. It is also possible that the folded, rolled up, bent, etc. substrate comprising the drug is filled inside of a capsule which can then be administered to a person in a conventional manner.

The shape of the medication after deformation may be, for instance, a tablet, a patch, a capsule, etc.

In the following, further exemplary embodiments of the medication will be explained. However, these also apply to the apparatus and to the method.

The solid carrier substrate may be biocompatible, particularly edible for a human being (or an animal). In this context, “edible” means that the solid carrier substrate can be eaten by a human patient without causing illness, poisoning, or any other physiological incompatibility with the human body. Thus, it is possible that the solid carrier substrate after deposition of the drug is administered to a person so that the drug can be released during digestion of the solid carrier substrate.

It is also possible that the medication is used as an orally applyable medication. Alternatively, it is possible to administer the medication in an anal way, such as with a suppository.

Alternatively, the medication may be adapted as a band aid (or a patch) adherable externally onto a skin portion of a human being (or an animal). In such an embodiment, the medication may have a strip-like arrangement, for instance to cover a wound. End portions of the band aid may comprise a glue portion for fixedly connecting the band aid to the skin of the human being (or an animal). As an addition to a protection function of such a band aid, a drug deposited to the band aid may also fulfill a medical task such as disinfection or promotion of healing.

The solid carrier substrate may comprise multiple layers, wherein different drugs may be provided on or in different ones of the multiple layers or between adjacent ones of the multiple layers. With such a stacked configuration, a sequence of physiological impacts of the various drugs may be selectively adjusted before administering the medication to a physiological object. Since drugs connected to or buried in layers will be exposed to the physiological subject later than layers exposed to a surface, an undelayed or a delayed release of drugs may be adjusted.

The solid carrier substrate may be absorbant.

The solid carrier substrate may comprise cellulose or any other polymer which can be decomposed by a metabolism of a human being (or an animal) and which is biocompatible and/or edible.

The solid carrier substrate may be adapted to be modified upon contact with a body fluid, particularly may be modified in one manner of the group consisting of being degraded upon contact with a body fluid, being digested upon contact with a body fluid, and being deformed upon contact with a body fluid.

The solid carrier substrate may be adapted to be modified upon contact with a body fluid, particularly may be degraded upon coming into contact with a body fluid or may be deformed (mechanically/geometrically) upon contact with a body fluid. This may then define a point of time at which the medication provides an impact to the physiological subject.

Alternatively, it is also possible that the solid carrier substrate maintains its shape upon contact with a body fluid.

The solid carrier substrate may comprise a plurality of separate sections being separated by a perforation (for instance a linear perforation line). A portion between two subsequent perforations may define one dose unit, for instance.

The solid carrier substrate may be planar.

It is also possible that some kind of paper may be used as a carrier substrate for a medication. The medication can be degraded upon contact with a body fluid (such as blood, urine, interstitial fluid, gastric acid, saliva, etc.), or may change its shape by moisture expansion and partial disintegration. Alternatively, the paper may maintain its initial shape even when being brought in contact with a body fluid. During a printing procedure, droplets may be formed from a solution which may be applied to the paper. The solvent may be removed after application, by drying. The drug may be deposited on the paper structure, or may be provided within the paper structure in a disperse distributed manner. The drug may be applied onto a paper stripe of any desired length, allowing for performing a continuous production. It is possible to apply a drug substance multiple times at the same position of the paper in order to thereby control the amount of drug per paper area. The medication can be formed in individual single doses by separating the paper strip at dedicated positions, allowing to define accurately individual doses or integer multiples thereof. The amount of the medication which is applied to the paper can be determined during the printing method using appropriate analytical methods.

The drug may be formed in individual doses, which can be combined to larger doses by separating the paper strip in specific sections in accordance with previously printed analysis and production parameters.

It is possible that, after drying and singularization, the manufactured medication can be combined to further shapes and forms of medications. This can be capsules, tablets, oral patches, etc. It is possible to insert the paper strip in a rolled or folded form into capsules. By the manner of folding and rolling of the paper strip and the sequence of the application procedure, it is possible to combine multiple drugs in one capsule which are separated by non-printed portions of the paper so that no physical, chemical, or other interactions occur due to a direct contact.

Due to the kind of folding and rolling of the paper strip and the sequence of the application procedure, it is possible to adjust the medication regarding its properties with respect to modified release of pharmaceutical agents, degradation, light protection, etc.

Performing such measures may allow to provide printed structures for intelligent medications, ensuring very precise dosing even on a picoliter scale. It also allows for the production of highly complex modular structures. Multiple agent/time release formulations are possible. Moreover, applications in the field of personalized medicine are possible.

In an embodiment, it is possible that one or more individual active pharmaceutical ingredients of the medication may be coated by a coating layer. Such a coating may be selected so that a retard effect in a body of a human being or an animal can be obtained, i.e. a delayed release of the active pharmaceutical ingredient into a physiological object. Additionally or alternatively, such a coating may be selected so that different active pharmaceutical ingredients can be applied to the substrate without undesired interactions.

In another embodiment, it is possible that a sufficient dispersion of the particles (for instance in an emulsion or a suspension) is promoted by integrating a shaking or vibrating mechanism in one or more containers. This may be done, for instance, with a mechanical, electrical or magnetic mechanism. Embodiments of the invention may allow to print pharmaceutical suspensions and emulsions even with particles being larger than 1 μm.

The aspects defined above and further aspects of the invention are apparent from the examples of embodiment to be described hereinafter and are explained with reference to these examples of embodiment.

### BRIEF DESCRIPTION OF THE DRAWINGS

The invention will be described in more detail hereinafter with reference to examples of embodiment but to which the invention is not limited.

FIG. 1 illustrates an apparatus for manufacturing a medication according to an exemplary embodiment of the invention.

FIG. 2 illustrates a cross-section of a medication according to an exemplary embodiment of the invention having a plurality of time release layers each including a specific drug.

FIG. 3 illustrates a band aid according to an exemplary embodiment of the invention having two different drugs.

FIG. 4 shows an apparatus for manufacturing such a medication according to an exemplary embodiment of the invention.

FIG. 5 to FIG. 7 illustrate an apparatus for manufacturing a medication in different operation states according to another exemplary embodiment of the invention.

FIG. 8 illustrates an apparatus for manufacturing a medication according to another exemplary embodiment of the invention.

FIG. 9 and FIG. 10 show a configuration of manufacturing a medication according to an exemplary embodiment of the invention.

FIG. 11 illustrates an alternative configuration of manufacturing an medication according to an embodiment of the invention.

FIG. 12 illustrates an apparatus for manufacturing a medication according to another exemplary embodiment of the invention.

### DETAILED DESCRIPTION

The illustrations in the drawings are shown schematically. In different drawings, similar or identical elements are provided with the same reference signs.

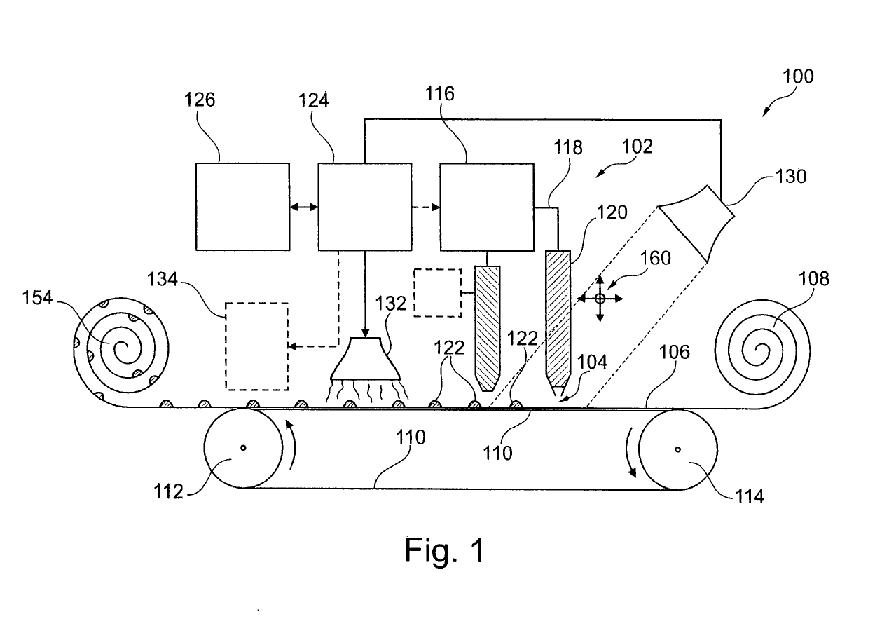

In the following, referring to FIG. 1, an apparatus 100 for manufacturing a medication according to an exemplary embodiment of the invention will be explained.

The apparatus 100 comprises an ejection unit 102 for ejecting a predefined amount (in the shown embodiment one droplet 104) of a drug against high blood pressure (or any other drug) onto a solid substrate 106 made of an edible paper (on a cellulose base).

As can be taken from FIG. 1, the edible paper 106 is rolled up on a source roll 108 and is conveyed by a conveyor belt 110 driven by two engine-operated drive rolls 112, 114. Preferably, one of the drive rolls 112, 114 is actively driven, the other one follows the motion with low friction. During the transport procedure, specific portions of the edible paper 106 are coming into functional contact with the ejector unit as will be explained in the following. After a drug 104 has been applied to the edible paper 106 in accordance with a specific protocol, the readily manufactured medication is rolled up on a destination roll 154.

More specifically, as can be taken from FIG. 1, the ejector unit 102 comprises a drug container 116 which accommodates a large amount of the drug. Furthermore, a conduit 118 connects the drug container 116 with an ejection nozzle 120 in which an amount of the drug is accommodated. Upon exerting pressure pulses on the nozzle 120, one or more droplets of the drug 104 are ejected via a front opening of the nozzle 120 and are directed onto a specific surface portion of the edible paper 106. As can be taken from FIG. 1, multiple spots 122 are provided on specific surface portions of the paper 106.

As indicated schematically in FIG. 1, further nozzles/further containers may be provided for applying the same or other drugs or also auxiliary components onto specific portions of the moving edible paper 106.

A control unit 124 (such as a microprocessor or a central processing unit (CPU)) controls operation of the container 116 and the nozzle 120 to eject the droplets 104 in accordance with an automatic or a user-defined protocol. Such a protocol may be defined by a user via an input/output unit 126 which is coupled for bidirectional communication with the control unit 124. The input/output unit 126 may include input elements such as buttons, a keypad, a joystick, etc. and may include an output unit such as a display via which a user may control progress of the manufacturing procedure.

However, control of container 116 by control unit 124 is merely optional. It is possible that the containment of the fluid is in contact with the surrounding pressure. The fluid may be taken from container 116 by being expelled via the nozzle 120. Hence, it is possible that the container is free of any control.

The control unit 124 may also move the nozzle 120 in any desired manner, for instance in a horizontal and/or vertical direction referring to FIG. 1, and/or in a direction perpendicular to a paper plane of FIG. 1 (as indicated by reference numeral 160). Thus, the drive system 112, 114, 110 for driving the paper 106 is optional and can be substituted or supplemented by a corresponding motion of the capillary 120 (for instance, it may be possible that a sheet of carrier substrate is mounted on an accommodation surface and is removed from the accommodation surface after having applied the drug to the sheet of carrier substrate; subsequently, a next sheet of carrier substrate may be mounted on the accommodation surface, and so on). The motion of the nozzle 120 in a vertical direction may support the deposition procedure, since the printing procedure may also be initiated by a deceleration force (or other forces) acting on the nozzle 120 upon abutting against the surface of the paper 106. By moving the nozzle 120 in a direction perpendicular to the paper plane of FIG. 1, the application of drug spot 120 is possible along an extension of the strip-like edible paper 106 perpendicular to the paper plane of FIG. 1.

However, when the volumes to be deposited are very small, it may happen that very large accelerations would be required for such a deposition procedure to overcome capillary forces. It is also possible that a motion of the nozzle 120 can bring a droplet attached to the nozzle 120 in contact with the substrate 106. The adhering forces at the substrate 106 may then be used for separating the droplet from the nozzle.

An optical monitoring unit 130 is provided as a CCD camera which detects the spots 122 after application onto the edible paper 106. In case that the monitoring unit 130 determines that a spot 122 has an undesired property (for instance a volume of the drug 104 being too small), the corresponding medication may either be classified as defective, or the spotting procedure may be repeated, for instance to add a lacking volume of the drug 104.

After having applied the spots 122 (comprising a solvent such as water or ethyl alcohol), a drying unit 132 dries the liquid spots 122 by the application of a stream of a heated gas. Thus, the drying unit 132 may be a hot fan which is operable under the control of the control unit 124.

After being dried by the fan 132 (or any other drying unit such as an electromagnetic radiation beam, for instance light), the sections of the edible paper 106 provided with the dried spots of drug may be perforated by a perforation/cutter unit 134 which includes perforations between adjacent sections of medications, thereby defining single doses thereof.

The control unit 124 may also serve as an identifier forming unit and may drive the nozzle 120 in such a manner that the nozzle 120 prints the drug spots 122 in a manner so as to form an inscription (for instance using the drug 104 as an ink) based on which the manufacturing history can be derived later.

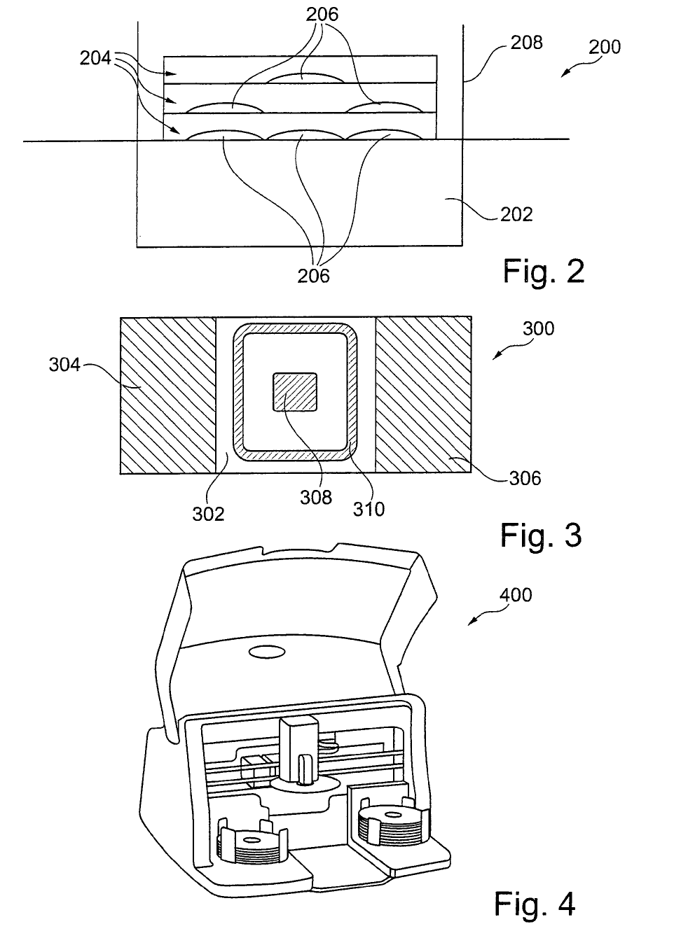

FIG. 2 illustrates a multiple layer medication according to an exemplary embodiment of the invention.

A carrier substrate 202 is provided, for instance made from an edible paper. Various additional layers 204 of edible paper are applied one after the other onto the carrier substrate 202. On a bottom portion of each of the additional layers 204, corresponding spots of medication 206 are formed. This medication may then be optionally inserted in a sheath or capsule 208.

When the medication 200 is administered to a patient, the human body starts decomposing the capsule 208 and subsequently the edible paper 202, 204, starting with exposed portions thereof. Therefore, at first the medication spot 206 included in a lower portion of the uppermost layer 204 is released to the body, followed by the two medication spots 206 in the middle layer, then followed by the three medication spots 206 in the lower layer 204. Thus, a time released intelligent medication is provided with the embodiment of FIG. 2. The medication 200 is configured for oral use.

In contrast to this, FIG. 3 shows a band aid 300 (or patch) as a medication according to another exemplary embodiment of the invention which is configured to be attached externally to a human body.

Again, a biocompatible solid carrier substrate 302 is foreseen which comprises a first glue portion 304 and a second glue portion 306 for connecting the band aid 300 to a skin portion of a person. In a central portion of the band aid 300 and on the biocompatible solid carrier substrate 302 a first central medication spot 308 and a surrounding second medication spot 310 are provided which are printed on the carrier substrate 302 in accordance with the scheme similar to the one shown in FIG. 1.

FIG. 4 is a three-dimensional view of an apparatus 400 for manufacturing a medication according to an exemplary embodiment of the invention.

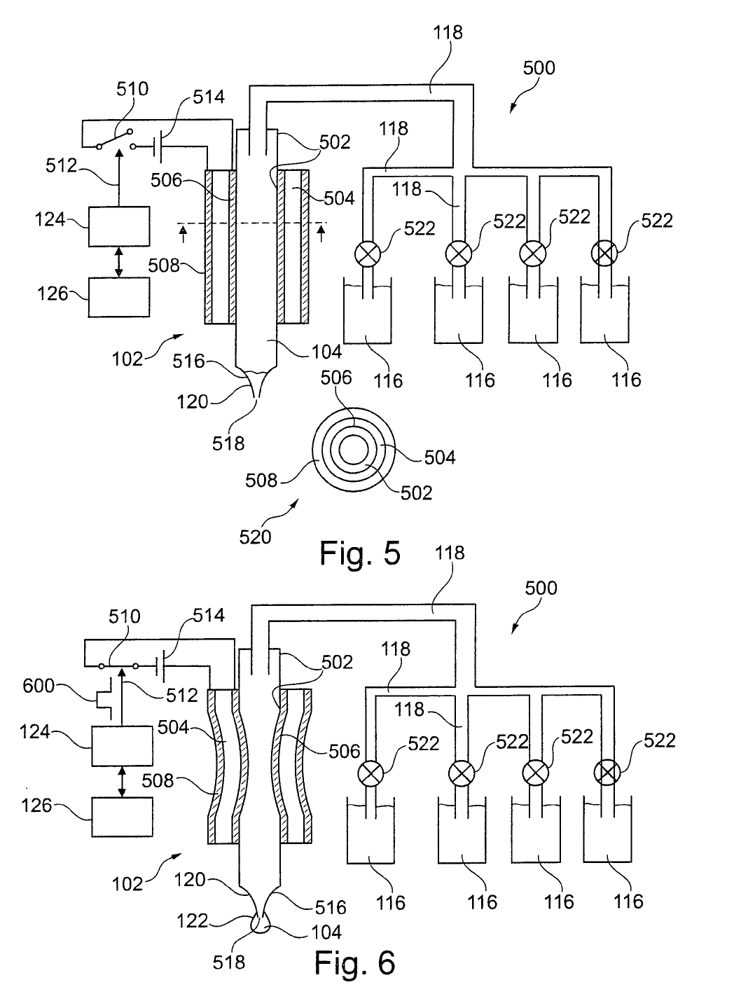

In the following, referring to FIG. 5 to FIG. 7, an apparatus 500 according to another exemplary embodiment of the invention will be explained.

Not all components of the apparatus 500 are shown in FIG. 5. All components which are shown in the embodiment of FIG. 1 can also be implemented according to FIG. 5. For example, the solid carrier substrate 106 and a means of transporting the latter is not shown in FIG. 5 but can be implemented in a similar manner as shown explicitly in FIG. 1. On the other hand, some of the components shown in FIG. 5 are not shown in FIG. 1 but can of course be implemented in this embodiment as well. For example, components related to a piezoelectric actuation which will be described in the following in more detail, can also be applied to the embodiment of FIG. 1.

The apparatus 500 shown in FIG. 5 is configured for manufacturing a medication. For this purpose, an ejection unit 102 is provided which is adapted for ejecting a predefined amount of a drug 104 which may have a liquid component to a solid carrier substrate (such as the solid carrier substrate 106 shown in FIG. 1 which would be located below the components shown explicitly in FIG. 5).

Furthermore, the apparatus 500 comprises a control unit 124, a capillary 502 and a tubular piezoelectric actuator 504 circumferentially covering side walls of the capillary 502 to form a concentric structure. The tubular piezoelectric actuator 504 surrounds a central portion of the capillary 502. A first tubular electrode 506 is located between the tubular capillary 502 and the tubular piezoelectric actuator 504. A second tubular electrode 508 is applied around an outer circumference or jacket of the tubular piezoelectric actuator 504. Reference numerals 502, 504, 506 and 508 form a concentric arrangement. In a cross-sectional view along line A-A′, these components have the appearance of four concentric circles (see reference numeral 520 in FIG. 5).

Electrodes 506, 508 are in electrical connection with an electric switch 510 (which can be realized as a transistor or a transistor circuitry). The control unit 124 is capable of applying a control signal 512 to the electric switch 510 to thereby selectively open or close electric switch 510. When the electric switch 510 is opened, as shown in FIG. 5, there is no current flow through the electrodes 506, 508 from a voltage source 514. Therefore, no electric signal is applied to the electrodes 506, 508 in the configuration of FIG. 5.

As can be taken from FIG. 5, the capillary 502 is tubularly shaped along a main portion thereof and has a tapering end 516 forming an orifice 518 via which the drug 104 can leave the capillary 502. Electrodes 506, 508 are provided as metallization layers applied to inner and outer surfaces, respectively, of the piezoelectric tube 504.

The embodiment of FIG. 5 furthermore shows that the capillary 502 is in fluid communication, via a bifurcated conduit 118, to a plurality of different drug containers 116. A predefined amount of a drug can be supplied from a respective one of the drug containers 116 in accordance with an opening degree of respective ones of the valves 522 shown in FIG. 5. By taking this measure, any desired drug mixture may be supplied to the capillary 502 for properly mixing before ejection via the orifice 518. Alternatively, individual ones of the drugs included in the drug containers 116 may be applied to the solid carrier substrate subsequently, i.e. one after the other.

FIG. 6 shows a scenario in which a high voltage pulse 600 has been applied via the electrodes 506, 508 to the piezoelectric actuator tube 504.

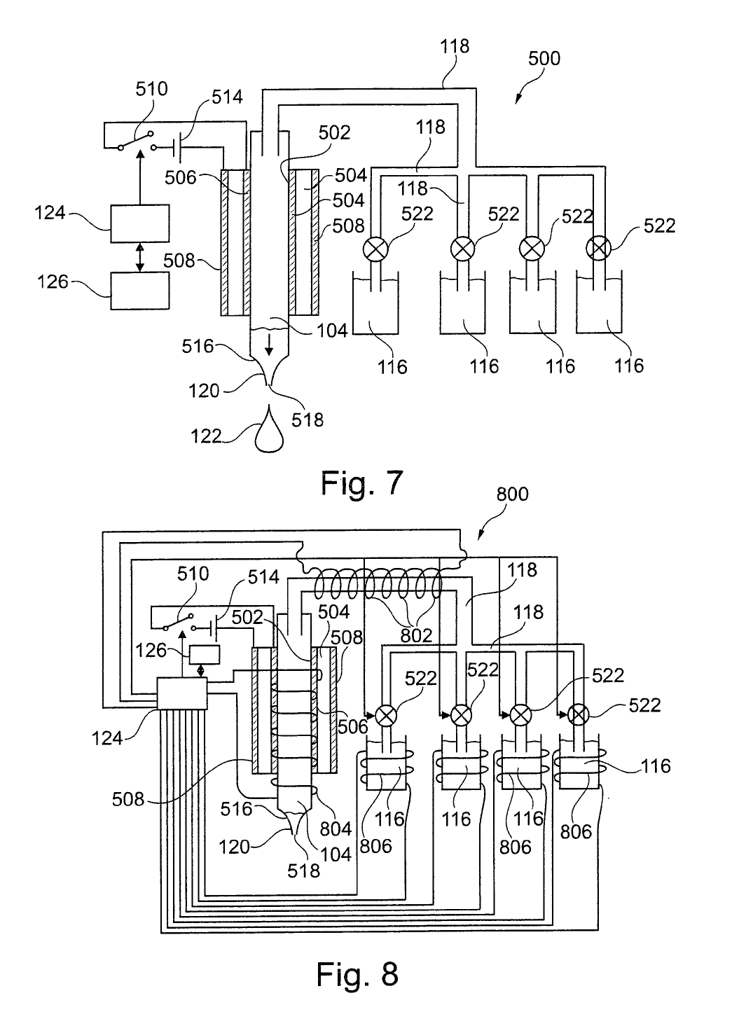

As can be taken from FIG. 6, upon applying a high voltage pulse 600 having a pulse length of for instance 100 μs to the electric switch 510, an electrical connection will be established between the voltage source 514 and the electrodes 506, 508 thereby activating the piezoelectric actuator tube 504. Consequently, a compression force will result in an inwardly bending of the piezoelectric actuator tube 504 which, in turn, results in an inward bending of a portion of the capillary 502 being laterally covered by the piezoelectric actuator 504. Hence, capillary 502 will be constricted. The piezoelectric actuator 504 will contract, thereby deforming the capillary 502 and generating an acoustic wave which is transmitted via the capillary 502 to the fluid of the drug 104. This acoustic wave propagates within the fluid of the drug 104 and may generate a motion of this fluid towards orifice 518. Without wishing to be bound to a specific theory, it is presently believed that this phase has only a duration of several microseconds. Subsequently, a deceleration of the fluid results due to a pressure drop. Consequently, a part of the ejected fluid volume will form a droplet 122, as can be taken from FIG. 6 and better from FIG. 7. Thus, in the scenario of FIG. 6, the high pressure pulse at the piezoelectric actuator 504 generates a pressure wave within the fluid 104. After switching off this pressure pulse by terminating the high voltage pulse 600, the piezoelectric actuator 504 relaxes, while the pressure wave propagates through the fluid of the drug 104.

The latter scenario is shown in FIG. 7 which further illustrates that droplet 122 is generated having left the orifice 518 and being directed with sufficient speed in a vertical direction parallel to gravity towards the solid carrier substrate (not shown in FIG. 7). The fluidic droplet 122 leaving the orifice 518 forms a freely propagating droplet 122.

Taking this measure allows to apply a droplet 122 of the drug 104 onto the solid carrier substrate 106 without direct mechanical contact between the tip at orifice 518 and the solid carrier substrate 106.

In an embodiment, it is possible to apply very small volumes of fluid between 30 pl and 500 pl with a volume variation of only few percent. Viscosities in a range between 1 mPas and 100 mPas can be used.

For instance, the diameter of the orifice 518 may be in a range between 20 μm to 100 μm. Such a dimension may be particularly suitable for drug applications. The volume of the droplet 122 may be defined basically by the diameter of the orifice 518 as well as by the viscosity of the fluid which, in turn, can be influenced by applying heat (for instance using components 802, 804 shown in FIG. 8) prior to the ejection of the fluid via the nozzle 518.

FIG. 8 shows an apparatus 800 according to another exemplary embodiment which is very similar to the apparatus shown in FIG. 5 to FIG. 7 but which has some specific features. These features can be applied in the embodiments of FIG. 1 and FIG. 5 to FIG. 7 as well.

In the embodiment of FIG. 8, a part of the fluid conduit 118 is surrounded by a spirally wound heating coil 802. Additionally, the portion of the capillary 502 being surrounded by the actuator 504 is also surrounded by an ohmic heating coil 804. Additionally, the containers 116 are also surrounded by an ohmic heating coil 806. Coils 802, 804, 806 are electrically connected to the control unit 124 so that the control unit 124 can apply heat to the fluid flowing through the conduit 118 or through the capillary 502 or in the containers 116, respectively, by conducting an electric current through one or more of the coils 802, 804, 806. Thus, it is possible to reduce the viscosity of the propagating fluid before ejection via the orifice 518 by heating. This allows to handle also highly viscous drugs with apparatus 800. For instance, drugs with a viscosity of up to 5000 mPas or more can be used with the embodiment of FIG. 8. However, these values are only examples and shall not limit the scope.

Also not shown in FIG. 5 to FIG. 8, it is possible to also use specific micropipettes according to an embodiment of the invention. A glass tip of such micropipettes (compare reference numerals 518, 120) can immerse directly into a fluid container 116, suck corresponding fluids with a low pressure within the capillary 502, move the corresponding pipettes using a (two-dimensional or three-dimensional) positioning system towards a substrate and then eject the drug 104 towards solid carrier substrate 106 using the piezoelectric actuation principle of FIG. 5 to FIG. 8.

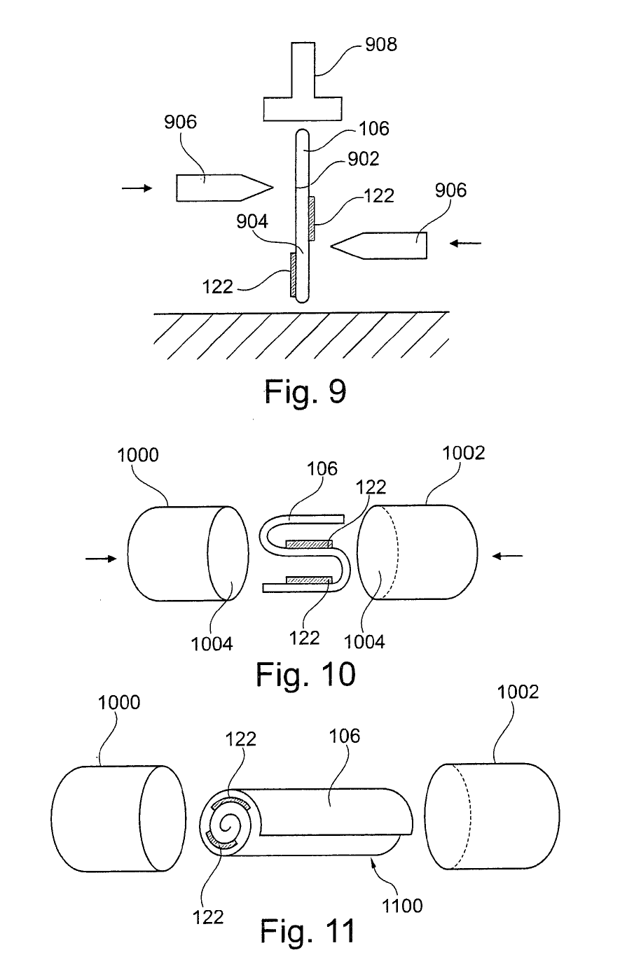

FIG. 9 and FIG. 10 show a post-processing of the manufactured drug, i.e. processes which can be performed after having applied the drug droplet 122 onto the edible paper 106.

As can be taken from FIG. 9, the basically planar solid carrier substrate 106 may be compressed, i.e. folded along folding lines 902, 904. In the embodiment of FIG. 9, these folding lines 902, 904 may be defined by a perforator or by a mandrel 906.

After having defined the folding lines 902, 904, a plunger 908 may bring the solid carrier substrate 106 in a configuration as shown in FIG. 10. In such a basically S-shaped folded configuration, a first capsule element 1000 (or a first shell element) and a correspondingly shaped and dimensioned second capsule element 1002 (or a second shell element) may receive the folded solid carrier substrate 106 with the applied medication in an accommodation volume 1004. Thus, after having connected the two capsule elements 1000, 1002 (see arrows in FIG. 10) the drug 122 with the edible paper 106 may be safely received within the capsule 1000, 1002 which can be a gelatine capsule.

FIG. 9 and FIG. 10 show substrate 106 having a medication 122 applied to both opposing surfaces thereof.

FIG. 11 shows another configuration in which the edible paper 106 with the applied drug 122 has been rolled to form a roll 1100 which can then be received in a space-efficient member in a similar way as shown in FIG. 10 using half capsules 1000, 1002.

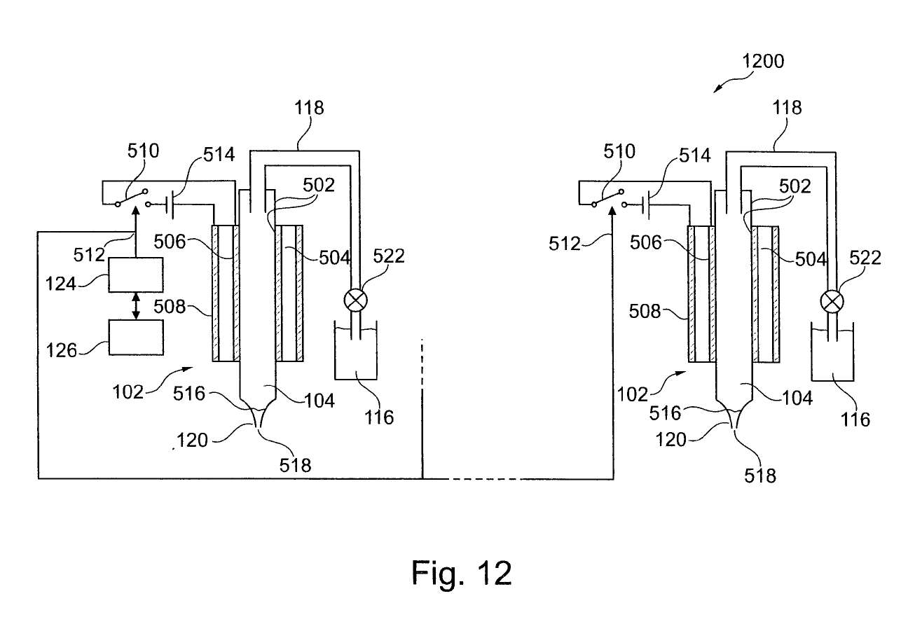

FIG. 12 illustrates an apparatus 1200 for manufacturing a medication according to another exemplary embodiment of the invention.

Apparatus 1200 is similar to apparatus 500 shown in FIG. 5. However, according to FIG. 12, each of a plurality of containers 116 (each holding a different fluid) has assigned a separate ejection unit 102. Although only two containers 116 are shown in FIG. 12, three or more containers 116 may be used as well. In the embodiment of FIG. 12, one control unit 124 is adapted to control each of the ejection units 102 individually. However, it is also possible that multiple control units 124 are provided, each of which being adapted to control a single ejection unit 102 or a group of ejection units 102.

In FIG. 12, each container 116 has assigned a separate connection tube 118 and a separate nozzle 120.

It should be noted that the term “comprising” does not exclude other elements or steps and the “a” or “an” does not exclude a plurality. Also elements described in association with different embodiments may be combined.

It should also be noted that reference signs in the claims shall not be construed as limiting the scope of the claims.

Implementation of the invention is not limited to the preferred embodiments shown in the figures and described above. Instead, a multiplicity of variants are possible which use the solutions shown and the principle according to the invention even in the case of fundamentally different embodiments.

1. An apparatus for manufacturing a medication, the apparatus comprising:
an ejector unit adapted for ejecting a predefined amount of a drug having a liquid component to a solid carrier substrate, wherein the ejector unit comprises a capillary and a tubular piezoelectric actuator surrounding at least a part of the capillary;
a control unit adapted for applying an electric signal to the piezoelectric actuator which, in response to the electric signal, is adapted to generate a compressional wave in the capillary for ejecting the predefined amount of the drug via an orifice of the capillary.

2.-4. (canceled)

5. The apparatus of claim 1, comprising a temperature adjustment unit adapted to adjust a temperature of the liquid comprising drug, particularly a heat application unit adapted to apply heat to the liquid comprising drug.

6. The apparatus of claim 5, wherein the temperature adjustment unit, particularly the heat application unit, is adapted to adjust a temperature of, particularly to apply heat to, the liquid comprising drug before leaving the capillary.

7.-8. (canceled)

9. The apparatus of claim 1, wherein the capillary has a tubular shape and is in fluid communication with one or more drug containers via a fluidic conduit.

10.-11. (canceled)

12. The apparatus of claim 1, wherein all components of the apparatus coming into contact with at least one of the drug and the solid carrier substrate are made of an inert material.

13. The apparatus of claim 1, further comprising a compression and encapsulation unit adapted for compressing a size of the solid carrier substrate after ejection of the predefined amount of the drug thereon and adapted for encapsulating the compressed solid carrier substrate within a capsule, particularly within a gelatine capsule.

14.-19. (canceled)

20. The apparatus of claim 1, wherein the control unit is adapted for controlling the ejector unit in accordance with a predefined or user-defined drug formation protocol and particularly is adapted for controlling at least one of the group consisting of a drug composition, an amount of a drug, a position of the solid carrier substrate to which the drug is to be ejected, and a geometrical shape of the applied drug.

21.-22. (canceled)

23. The apparatus of claim 1, comprising a monitoring unit, particularly an optical monitoring unit, adapted for monitoring the drug ejected onto the solid carrier substrate.

24. The apparatus of claim 1, comprising a singularization unit adapted for a singularization, particularly for cutting or perforating, the solid carrier substrate into separation sections each comprising a drug.

25. The apparatus of claim 24, wherein the singularization unit is adapted for singularizing the solid carrier substrate into separation sections of variable sizes.

26. The apparatus of claim 1, comprising an identifier forming unit adapted for forming, particularly for printing, an identifier onto the solid carrier substrate indicative of the ejected drug.

27. The apparatus of claim 26, wherein the identifier forming unit is adapted for forming the identifier by the drug itself.

28. The apparatus of claim 1, comprising a drying unit adapted for drying the drug by at least partially removing the liquid component after provision to the solid carrier substrate.

29. The apparatus of claim 1, comprising an auxiliary substance provision unit adapted for adding an auxiliary substance to the drug.

30. The apparatus of claim 29, wherein the auxiliary substance comprises at least one of the group consisting of an agent for modifying a release of an active component of the drug, an agent for modifying a degradation of the solid carrier substrate, and an agent for increasing a stability of an active component of the drug.

31. A method of manufacturing a medication, the method comprising:
providing a piezoelectric actuator surrounding at least a part of a capillary;
ejecting a predefined amount of a drug having a liquid component to a solid carrier substrate by applying an electric signal to the piezoelectric actuator so that the piezoelectric actuator, in response to the electric signal, generates a compressional wave in the capillary to thereby eject the predefined amount of the drug via an orifice of the capillary.

32.-33. (canceled)

34. The method of claim 31, wherein a shape of the medication is deformed after ejecting the drug to the solid carrier substrate.

35. The method of claim 34, wherein the shape of the medication is deformed to form a medication of one of the group consisting of a tablet, a patch, a suppository, and a capsule.

36. The method of claim 31 comprising folding or rolling the medication and inserting the medication into a shell.

37. The apparatus of claim 1, the apparatus comprising:
a solid carrier substrate, wherein a predefined amount of the drug ejected with the liquid component to the solid carrier substrate by the ejector unit.

38.-47. (canceled)
